(19) United States
(12) Patent Application Publication
(10) Pub. No.: US 2021/0287634 A1
(43) Pub. Date: Sep. 16, 2021

(54) MEDICAL IMAGE PROCESSING DEVICE, MEDICAL OBSERVATION SYSTEM, AND METHOD OF OPERATING MEDICAL IMAGE PROCESSING DEVICE

(71) Applicant: Sony Olympus Medical Solutions Inc., Tokyo (JP)

(72) Inventor: Tatsuya DEGUCHI, Tokyo (JP)

(73) Assignee: Sony Olympus Medical Solutions Inc., Tokyo (JP)

(21) Appl. No.: 17/147,553

(22) Filed: Jan. 13, 2021

(30) Foreign Application Priority Data
Mar. 16, 2020 (JP) 2020-045812

(51) Int. Cl. G09G 5/391; G06F 3/14; G06F 3/00; A61B 1/00

(52) U.S. Cl. CPC G09G 5/391; G06F 3/1454; G09G 2340/0407; A61B 1/00009; A61B 1/00016; G06F 3/002

(57) ABSTRACT

A medical image processing device includes an image processor configured to generate a display image based on a distance between a display device and a target and a size of a display screen in the display device, the display image being displayed on the display device. An image quality of the display image is lowered as the distance becomes larger and the size of the display screen becomes smaller.

### CROSS-REFERENCE TO RELATED APPLICATION

This application claims priority from Japanese Application No. 2020-045812, filed on Mar. 16, 2020, the contents of which are incorporated by reference herein in its entirety.

### BACKGROUND

The present disclosure relates to a medical image processing device, a medical observation system, and a method of operating a medical image processing device.

In the medical field and the industrial field, medical devices such as an endoscope device that captures a subject image using an imaging element and a medical microscope device are known (see, for example, JP 2016-209542 A). Among these, the endoscope device includes, for example, an endoscope, an imaging device, a display device, a control device, and a light source device. In the endoscope device, illumination light is supplied from the light source device via a light guide connected to the endoscope, and the illumination light is emitted to capture the subject image. An image captured by the endoscope device is displayed on the display device.

### SUMMARY

The amount of data of a display image has also increased along with an increase in the number of pixels of display devices. As the amount of data increases, the resolution of an image increases so that the visibility improves, but a load on data transmission and data processing increases.

There is a need for a medical image processing device, a medical observation system, and a method of operating a medical image processing device capable of suppressing a load related to data processing while maintaining the visibility.

According to one aspect of the present disclosure, there is provided a medical image processing device including an image processor configured to generate a display image based on a distance between a display device and a target and a size of a display screen in the display device, the display image being displayed on the display device, wherein an image quality of the display image is lowered as the distance becomes larger and the size of the display screen becomes smaller.

### BRIEF DESCRIPTION OF THE DRAWINGS

FIG. 1 is diagram illustrating a schematic configuration of an endoscope device according to a first embodiment;

FIG. 2 is a block diagram illustrating configurations of a camera head, a control device, and a light source device;

FIG. 3 is a view (Part 1) for describing a use mode of the endoscope device according to the first embodiment;

FIG. 4 is a view (Part 2) for describing the use mode of the endoscope device according to the first embodiment;

FIG. 5 is a block diagram illustrating configurations of a camera head, a control device, and a light source device of an endoscope device according to a first modification of the first embodiment;

FIG. 6 is a block diagram illustrating configurations of a camera head, a control device, and a light source device of an endoscope device according to a second modification of the first embodiment;

FIG. 7 is a view for describing a use mode of a microscope device of a medical observation system according to the second modification of the first embodiment;

FIG. 8 is a diagram illustrating a schematic configuration of an endoscope system according to a second embodiment; and

FIG. 9 is a diagram illustrating a schematic configuration of an operating microscope system according to a third embodiment.

### DETAILED DESCRIPTION

Hereinafter, a description will be given regarding modes for embodying the present disclosure (hereinafter, referred to as “embodiments”). A description will be given in the embodiments regarding a medical endoscope system that displays an in-vivo image of a subject, such as a patient, as an example of a medical observation system which includes a medical image processing device according to the present disclosure. In addition, the present disclosure is not limited to these embodiments. Further, the same reference sign will be assigned to the same components in the description of the drawings.

### First Embodiment

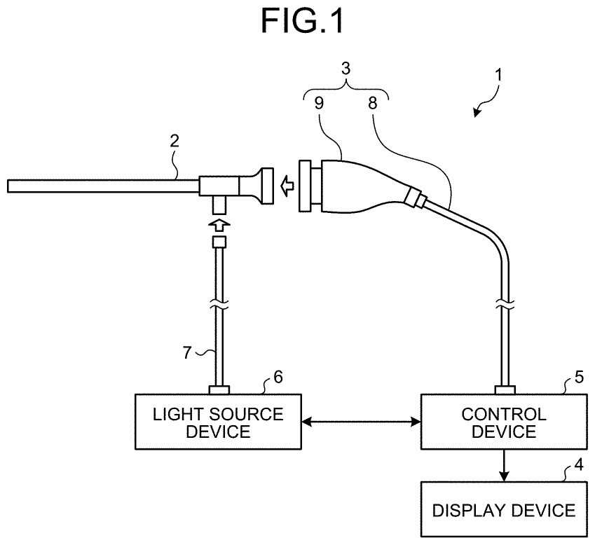

FIG. 1 is a diagram illustrating a schematic configuration of an endoscope system 1 according to a first embodiment. The endoscope system 1 is a device which is used in the medical field and observes an (in-vivo) subject inside an object to be observed such as human. As illustrated in FIG. 1, this endoscope system 1 is provided with an endoscope 2, an imaging device 3, a display device 4, a control device 5, and a light source device 6, and a medical observation system is constituted by the imaging device 3 and the control device 5.

The light source device 6 is connected with one end of a light guide 7, and supplies white light to the one end of the light guide 7 to illuminate the inside of a living body. Incidentally, the light source device 6 and the control device 5 may be configured as separate bodies communicating with each other as illustrated in FIG. 1, or may be integrated.

The one end of the light guide 7 is detachably connected with the light source device 6, and the other end thereof is detachably connected with the endoscope 2. Further, the light guide 7 transmits the light supplied from the light source device 6 from the one end to the other end, and supplies the light to the endoscope 2.

The imaging device 3 captures a subject image from the endoscope 2, and outputs the imaging result. As illustrated in FIG. 1, the imaging device 3 is provided with a transmission cable 8, which is a signal transmission unit, and a camera head 9. In the first embodiment, a medical imaging device is constituted by the transmission cable 8 and the camera head 9.

The endoscope 2, which is rigid and has an elongated shape, is inserted into the living body. An observation optical system, which includes one or a plurality of lenses and condenses the subject image, is provided inside the endoscope 2. The endoscope 2 emits the light supplied via the light guide 7 from a distal end thereof, and irradiates the inside of the living body with the emitted light. Then, the light with which the inside of the living body is irradiated (the subject image) is condensed by the observation optical system (a lens unit 91) inside the endoscope 2.

The camera head 9 is detachably connected with a proximal end of the endoscope 2. Further, the camera head 9 captures the subject image condensed by the endoscope 2 and outputs an imaging signal generated by the imaging, under the control of the control device 5. Incidentally, a detailed configuration of the camera head 9 will be described later. The endoscope 2 and the camera head 9 may be detachably configured as illustrated in FIG. 1, or may be integrated.

The transmission cable 8 has one end which is detachably connected with the control device 5 via a connector, and the other end which is detachably connected with the camera head 9 via the connector. To be specific, the transmission cable 8 is a cable which includes a plurality of electrical wirings (not illustrated) arranged inside an outer cover serving as the outermost layer. The plurality of electrical wirings is electrical wirings which are configured to transmit the imaging signal output from the camera head 9 to the control device 5, a control signal output from the control device 5, a synchronization signal, a clock, and power to the camera head 9.

The display device 4 displays an image generated by the control device 5 under the control of the control device 5. The display device 4 is connected to the control device 5 by a video cable (not illustrated). The display device 4 preferably includes a display unit which is equal to or larger than 55 inches, in order to easily obtain the sense of immersion during the observation, but is not limited thereto.

The control device 5 processes the imaging signal input from the camera head 9 via the transmission cable 8, outputs the image signal to the display device 4, and collectively controls the operation of the camera head 9 and the display device 4. Incidentally, a detailed configuration of the control device 5 will be described later.

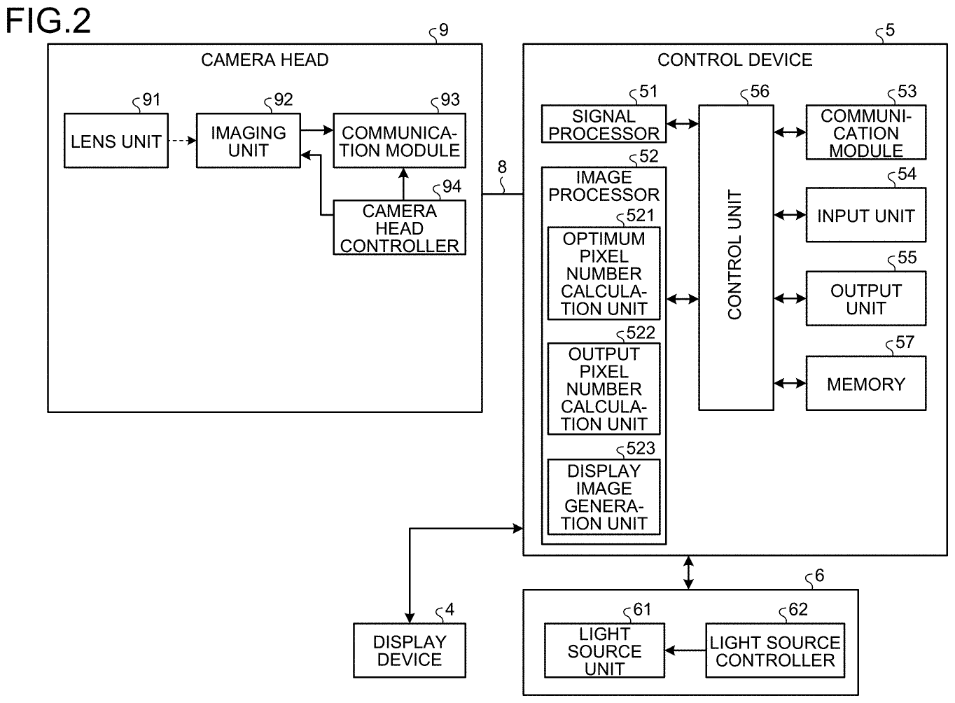

Next, a description will be given regarding each configuration of the imaging device 3, the control device 5, and the light source device 6. FIG. 2 is a block diagram illustrating configurations of the camera head 9, the control device 5, and the light source device 6. Incidentally, FIG. 2 does not illustrate the connector which makes the camera head 9 and the transmission cable 8 detachable from each other.

Hereinafter, the configuration of the control device 5 and the configuration of the camera head 9 will be described in the order. Incidentally, the main section of the present disclosure will be mainly described as the configuration of the control device 5, in the following description. As illustrated in FIG. 2, the control device 5 includes a signal processor 51, an image processor 52, a communication module 53, an input unit 54, an output unit 55, a control unit 56, and a memory 57. Incidentally, the control device 5 may be provided with a power supply unit (not illustrated) and the like, which generates a power-supply voltage to drive the control device 5 and the camera head 9, supplies the voltage to each unit of the control device 5, and supplies the voltage to the camera head 9 via the transmission cable 8.

The signal processor 51 performs noise removal, or signal processing such as A/D conversion, if necessary, with respect to the imaging signal output from the camera head 9, thereby outputting the digitized imaging signal (pulse signal) to the image processor 52.

In addition, the signal processor 51 generates the synchronization signal and the clock of the imaging device 3 and the control device 5. The synchronization signal (for example, a synchronization signal to instruct an imaging timing of the camera head 9, and the like) and the clock (for example, a clock for serial communication) with respect to the imaging device 3 are sent to the imaging device 3 via a line (not illustrated). The imaging device 3 is then driven based on the synchronization signal and the clock.

The image processor 52 generates an image signal for display, which is displayed by the display device 4, based on the imaging signal input from the signal processor 51. The image processor 52 includes an optimum pixel number calculation unit 521, an output pixel number calculation unit 522, and a display image generation unit 523.

The optimum pixel number calculation unit 521 calculates the optimum number of pixels according to the display device 4 to be used, based on a viewing distance and the limiting resolution of a human eye.

The viewing distance corresponds to a distance between a user (here, a surgeon) and the display device 4. In the first embodiment, the viewing distance is input via the input unit 54 and stored in the memory 57.

The limiting resolution is a value determined based on known spatial frequency characteristics of vision. In general, it is said that the number of stripes that may recognize a contrast difference of a vertical stripe pattern is most visible in the vicinity of 5 pairs per viewing angle of 1°, and the visibility deteriorates when exceeding 30 pairs (cycles/degree) particularly on the high frequency side. In the first embodiment, the limiting resolution F_{eye_lim} of the human eye is set in a range of, for example, 20≤F_{eye_lim}≤30 (cycles/degree).

The optimum pixel number calculation unit 521 calculates, for example, the number of pixels optimum for viewing based on the limiting resolution of the human eye and the viewing distance of the user (optimum number of pixels). A value of the optimum number of pixels decreases as the distance between the user and the display device 4 increases. Incidentally, the optimum number of pixels changes substantially depending on the viewing distance since the limiting resolution is a preset value.

The output pixel number calculation unit 522 calculates the number of output pixels suitable for an image to be displayed on the display device 4 based on the optimum number of pixels calculated by the optimum pixel number calculation unit 521 and a size of a display screen of the display device 4. Here, the size of the display screen is determined by a type of the display device 4 to be used. The size of the display screen is input via the input unit 54, or the information thereof is acquired from the display device 4 connected by the video cable. A value of the number of output pixels decreases as the optimum number of pixels decreases on the same display screen. As the number of output pixels decreases, the image quality of the image displayed on the display device 4 also deteriorates.

The display image generation unit 523 generates an image signal for display, which is displayed by the display device 4, based on the imaging signal input from the signal processor 51. The display image generation unit 523 generates the image signal for display including the subject image by executing predetermined signal processing with respect to the imaging signal. Here, the display image generation unit 523 performs known image processing such as detection processing and various types of image processing such as interpolation processing, color correction processing, color enhancement processing, noise reduction processing, and contour enhancement processing, and also generates an image signal to form an image of the number of pixels corresponding to the number of output pixels calculated by the output pixel number calculation unit 522.

Here, the image displayed on the display device 4 is expressed by a plurality of beams of light (dots) on the screen. In the first embodiment, the number of dots corresponds to the number of output pixels. The dot is constituted by pixels in the display device 4. The resolution of the image displayed by the display device 4 changes depending on the number of pixels constituting one dot.

The display image generation unit 523 generates the image corresponding to the number of output pixels by setting the number of pixels constituting one dot based on the number of output pixels. As the number of output pixels is smaller, the generated image becomes an image having lower resolution and lower image quality. The display image generation unit 523 outputs the generated image signal to the display device 4.

The communication module 53 outputs a signal, which includes a control signal to be described later that is transmitted from the control unit 56, from the control device 5, to the imaging device 3. In addition, the communication module 53 outputs a signal from the imaging device 3 to each unit in the control device 5. That is, the communication module 53 is a relay device that collects signals from the respective units of the control device 5, which are output to the imaging device 3, by, for example, parallel-to-serial conversion or the like, and outputs the collected signal, and that divides the signal input from the imaging device 3 by, for example, serial-to-parallel conversion or the like, and outputs the divided signals to the respective units of the control device 5.

The input unit 54 is implemented using a user interface such as a keyboard, a mouse, and a touch panel, and receives the input of various types of information.

The output unit 55 is implemented using a speaker, a printer, a display, and the like, and outputs various types of information. Under the control of the control unit 56, the output unit 55 outputs an alarm sound and an alarm light, and displays an image. Incidentally, the image generated by the camera head 9 is mainly displayed on the display device 4.

The control unit 56 performs drive control of the respective components including the control device 5 and the camera head 9, and input and output control of the information with respect to the respective components. The control unit 56 generates the control signal by referring to communication information data (for example, format information for communication and the like), which is recorded in the memory 57, and transmits the generated control signal to the imaging device 3 via the communication module 53. In addition, the control unit 56 outputs the control signal to the camera head 9 via the transmission cable 8.

The memory 57 is implemented using a semiconductor memory such as a flash memory and a dynamic random access memory (DRAM). In the memory 57, the communication information data (for example, the format information for communication and the like) is recorded. Incidentally, various types of programs to be executed by the control unit 56 and the like may be recorded in the memory 57.

Incidentally, the signal processor 51 may include an AF processor, which outputs a predetermined AF evaluation value of each frame based the input imaging signals of the frames, and an AF calculation unit which performs AF calculation processing to select a focus lens position or the like that is the most suitable as a focusing position, from the AF evaluation values of the respective frames output from the AF processor.

The signal processor 51, the image processor 52, the communication module 53, and the control unit 56, described above, are implemented using a general-purpose processor such as a central processing unit (CPU) including an internal memory (not illustrated) in which the program is recorded, or dedicated processors including various types of arithmetic circuits and the like which execute specific functions such as an application specific integrated circuit (ASIC). In addition, they may include a field programmable gate array (FPGA) (not illustrated) which is one type of a programmable integrated circuit. Incidentally, in the case of including the FPGA, the FPGA, which is the programmable integrated circuit, may be configured by providing a memory to store configuration data, and using the configuration data read out from the memory.

The light source device 6 has a light source unit 61 which is connected with one end of the light guide 7 and supplies, for example, white light for illuminating the inside of the living body to the one end of the light guide 7, and a light source controller 62 that controls the emission of the illumination light of the light source unit 61. Incidentally, the light source device 6 and the control device 5 may be configured as separate bodies communicating with each other as illustrated in FIG. 1, or may be integrated.

The light source unit 61 is configured using, for example, any of a xenon light source, an LED light source, a laser light source, and a projector light source. Incidentally, the projector light source includes a light source that emits white light, a projection element such as a digital mirror device (DMD) and a liquid crystal panel, and an optical system that emits the projected light to the outside.

The light source controller 62 causes the light source to emit the white light based on an instruction from the control device 5.

Next, the main section of the present disclosure will be mainly described as the configuration of the camera head 9. As illustrated in FIG. 2, the camera head 9 is provided with a lens unit 91, an imaging unit 92, a communication module 93, and a camera head controller 94.

The lens unit 91 is configured using one or a plurality of lenses, and forms the subject image passing through the lens unit 91 on an imaging surface of an imaging element constituting the imaging unit 92. The one or the plurality of lenses is configured to be movable along the optical axis. Further, the lens unit 91 is provided with an optical zoom mechanism (not illustrated) that changes an angle of view by moving the one or the plurality of lenses, and a focus mechanism that changes a focal point position. Incidentally, the lens unit 91 forms an observation optical system that guides observation light incident on the endoscope 2 to the imaging unit 92 together with the optical system provided in the endoscope 2.

The imaging unit 92 captures a subject image under the control of the camera head controller 94. The imaging unit 92 is configured using an imaging element that receives the subject image formed by the lens unit 91 and converts the subject image into an electrical signal. The imaging element is configured using a charge coupled device (CCD) image sensor or a complementary metal oxide semiconductor (CMOS) image sensor. For example, when the imaging element is the CCD, a signal processor (not illustrated), which performs signal processing (A/D conversion or the like) with respect to an electrical signal (analog signal) from the imaging element to output an imaging signal, is mounted to a sensor chip or the like. For example, when the imaging element is the CMOS, a signal processor (not illustrated), which performs signal processing (A/D conversion or the like) with respect to an electrical signal (analog signal) obtained by converting light into the electrical signal to output the imaging signal, is included in the imaging element. The imaging unit 92 outputs the generated electrical signal to the communication module 93. Incidentally, when the projector light source is used, it is preferable that the number of pixels of the image sensor of the imaging unit 92 and the number of pixels of the projection element of the light source unit 61 be the same.

The communication module 93 outputs the signal transmitted from the control device 5 to the respective units inside the camera head 9 such as the camera head controller 94. In addition, the communication module 93 converts the information relating to a current state of the camera head 9 into a signal format according to a transmission scheme which has been set in advance, and outputs the converted signal to the control device 5 via the transmission cable 8. That is, the communication module 93 is a relay device that divides the signal input from the control device 5 and the transmission cable 8 by, for example, the serial-to-parallel conversion or the like and outputs the divided signals to the respective units of the camera head 9, and that collects signals from the respective units of the camera head 9 output to the control device 5 and the transmission cable 8 by, for example, the parallel-to-serial conversion or the like and outputs the collected signal.

The camera head controller 94 controls the operation of the entire camera head 9 according to a drive signal input via the transmission cable 8 and an instruction signal output from an operating unit when the user operates the operating unit, such as a switch, which is provided to be exposed on an external surface of the camera head 9. In addition, the camera head controller 94 outputs the information relating to the current state of the camera head 9 to the control device 5 via the transmission cable 8.

Incidentally, the communication module 93 and the camera head controller 94 described above are implemented using a general-purpose processor such as a CPU having an internal memory (not illustrated) in which a program is recorded or a dedicated processor such as various arithmetic circuits that execute a specific function such as an ASIC. In addition, they may be configured using the FPGA which is one type of the programmable integrated circuit. Here, in the case of including the FPGA, the FPGA, which is the programmable integrated circuit, may be configured by providing a memory to store configuration data, and using the configuration data read out from the memory.

In addition, the camera head 9 and the transmission cable 8 may include a signal processor which executes signal processing with respect to the imaging signal generated by the communication module 93 or the imaging unit 92. Further, an imaging clock to drive the imaging unit 92 and a control clock for the camera head controller 94 may be generated based on a reference clock generated by an oscillator (not illustrated) provided inside the camera head 9, and be output to the imaging unit 92 and the camera head controller 94, respectively. Alternatively, timing signals for various types of processing in the imaging unit 92 and the camera head controller 94 may be generated based on the synchronization signal input from the control device 5 via the transmission cable 8 and be output to each of the imaging unit 92 and the camera head controller 94. In addition, the camera head controller 94 may be provided not in the camera head 9, but in the transmission cable 8 or the control device 5.

In the endoscope system 1 described above, the image based on the electrical signal captured by the imaging unit 92 is displayed on the display device 4, and the feedback control of the light source device 6 is performed based on the image signal displayed on the display device 4.

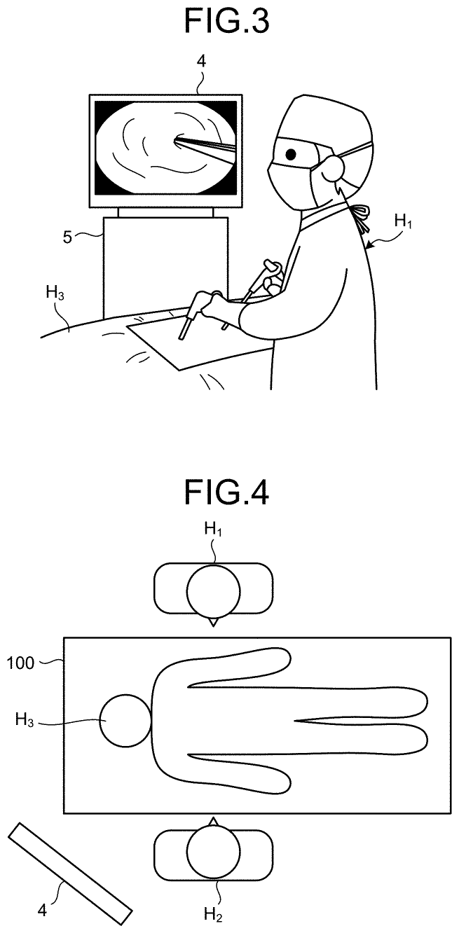

FIGS. 3 and 4 are views (Part 1) for describing a use mode of the endoscope device according to the first embodiment. Incidentally, FIG. 4 illustrates a situation during the treatment as viewed from directly above. A surgeon H₁ performs an operation while observing a video of a surgical site displayed on the display device 4. The surgeon H₁ uses the endoscope 2 to perform the operation on a patient H₃ lying on an operating table 100. In addition, FIG. 4 illustrates not only the surgeon H₁ who performs the operation but also an assistant H₂ who assists the operation. Incidentally, the arrangement in which the display device 4 is installed to be positioned substantially in front of the surgeon H₁ when performing the operation in a standing position is illustrated in the present embodiment. In the example illustrated in FIG. 4, the viewing distance corresponds to the distance between the surgeon H₁ and the display device 4. The viewing distance may be set based on the position where the surgeon H₁ may stand based on the arrangement of the operating table 100 and the display device 4 or may be set individually for each surgeon H₁.

In the first embodiment described above, output pixels are set according to the distance (viewing distance) between the surgeon and the display device 4, and an image having the resolution corresponding to the output pixels is displayed on the display device 4. At this time, the resolution decreases to such an extent that the visibility is maintained as the distance between the surgeon and the display device 4 increases. When the resolution decreases, the amount of data of an image signal transmitted from the control device 5 to the display device 4 is reduced, and a load related to transmission and processing may be reduced. On the other hand, when an attempt is made to maintain the visibility regardless of the distance between the surgeon and the display device 4, a high-resolution image is regularly generated so that the load related to processing and transmission is particularly large for a display device having a large number of pixels. According to the first embodiment, the image whose resolution has been lowered according to the distance is generated. Thus, the amount of data is reduced to such an extent that the visibility is maintained when the distance is long, so that the load related to the data processing may be suppressed while maintaining the visibility.

Incidentally, in the first embodiment, when the image generated by the display image generation unit 523 is stored in the memory 57, an image with the maximum number of pixels in the display device 4 may be stored, an image according to the number of output pixels generated by the display image generation unit 523 for display may be stored, or both the images may be stored.

### First Modification of First Embodiment

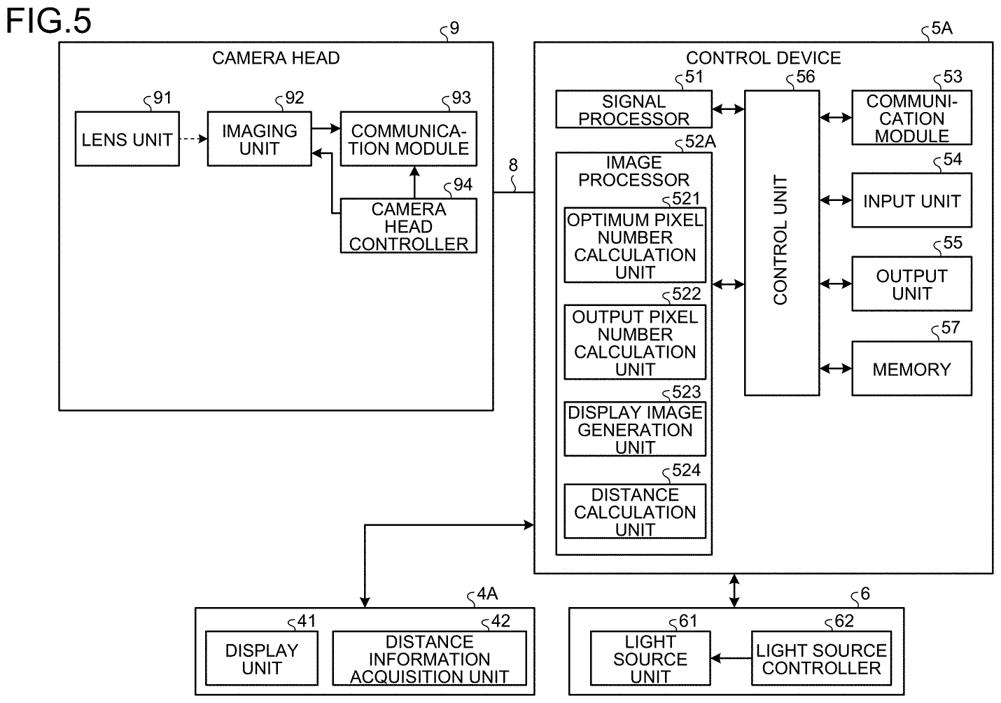

Next, a first modification of the first embodiment will be described. FIG. 5 is a block diagram illustrating configurations of a camera head, a control device, and a light source device of an endoscope device according to the first modification of the first embodiment. Incidentally, the same components as those in the first embodiment described above are denoted by the same reference signs.

The endoscope device according to the first modification includes the endoscope 2, the imaging device 3, a display device 4A, a control device 5A, and the light source device 6.

The display device 4A has a display unit 41 and a distance information acquisition unit 42. The display device 4A is connected to the control device 5 by a video cable (not illustrated).

The display unit 41 displays an image or the like.

The distance information acquisition unit 42 acquires information on a distance between the display device 4A and a measurement target (here, a surgeon). The distance information acquisition unit 42 may use a known distance measuring means such as a distance measuring sensor that generates distance information to generate the distance between the measurement target and the display device 4A. The distance information acquisition unit 42 acquires information on a preset target, for example, the distance to the surgeon H₁ illustrated in FIG. 4. Incidentally, when there is a plurality of people who are likely to serve as measurement targets in a distance-measurable range, information on the distance to each person may be generated for each person.

The control device 5A includes the signal processor 51, an image processor 52A, the communication module 53, the input unit 54, the output unit 55, the control unit 56, and the memory 57.

The image processor 52A generates an image signal for display, which is displayed by the display device 4A, based on an imaging signal input from the signal processor 51. The image processor 52 includes the optimum pixel number calculation unit 521, the output pixel number calculation unit 522, the display image generation unit 523, and a distance calculation unit 524.

The distance calculation unit 524 calculates the distance between the display device 4 and the surgeon based on the distance information acquired from the distance information acquisition unit 42. The distance calculation unit 524 outputs the calculated distance to the optimum pixel number calculation unit 521.

Here, when the distance calculation unit 524 acquires the distance information on a plurality of people, the distance calculation unit 524 calculates the distance according to set conditions. For example, the distance of a person to be controlled is calculated, or the distance to the closest person is calculated. The optimum pixel number calculation unit 21 calculates the optimum number of pixels based on the distance to the closest person, so that an image in which the visibility is maintained even for the closest person is generated.

The optimum pixel number calculation unit 521 calculates the optimum number of pixels based on the limiting resolution and the distance acquired from the distance calculation unit 524.

Thereafter, the output pixel number calculation unit 522 calculates the number of output pixels, and the display image generation unit 523 generates an image signal for display in the same manner as in the first embodiment.

According to the first modification described above, the same effect as that of the first embodiment may be obtained. Further, the distance between the display device 4A and the surgeon is calculated based on the information obtained by distance measurement in the first modification, and thus, it is possible to implement the image generation processing based on a more accurate distance as compared with the case where the distance is set in advance based on the position of the display device.

Further, according to the first modification, the distance information acquisition unit 42 is made to periodically generate distance information, and output pixels of an image are set each time the distance information is input, so that it is possible to display the image having the resolution that follows the movement of the surgeon.

### Second Modification of First Embodiment

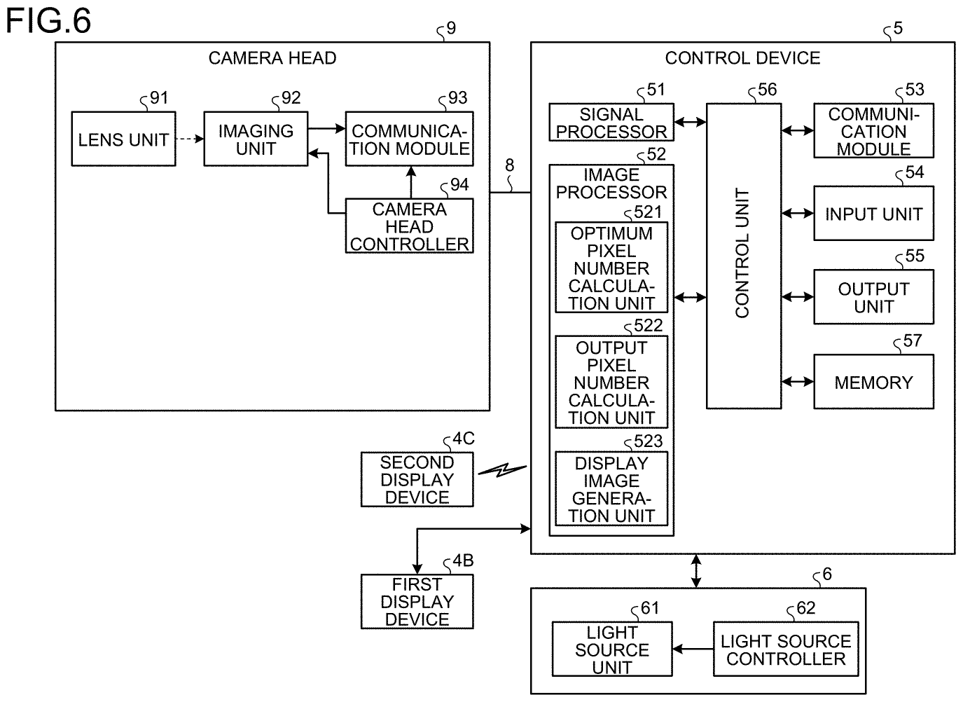

A second modification of the first embodiment will be described. FIG. 6 is a block diagram illustrating configurations of a camera head, a control device, and a light source device of an endoscope device according to the second modification of the first embodiment. Incidentally, the same components as those in the first embodiment described above are denoted by the same reference signs.

The endoscope device according to the second modification includes the endoscope 2, the imaging device 3, a first display device 4B, a second display device 4C, the control device 5, and the light source device 6.

The first display device 4B displays an image generated by the control device 5 under the control of the control device 5. The first display device 4B is connected to the control device 5 by a video cable (not illustrated).

The second display device 4C displays an image generated by the control device 5 under the control of the control device 5. The second display device 4C transmits and receives a signal to and from the control device 5 by wireless communication.

The control device 5 includes the signal processor 51, the image processor 52, the communication module 53, the input unit 54, the output unit 55, the control unit 56, and the memory 57. In the second modification, the control device 5 controls the camera head 9 and the light source device 6 as described above, and also controls display of the first display device 4B and the second display device 4C. Incidentally, in the second modification, the distance between an operating table (or a position where a surgeon may stand) and the first display device 4B, and the distance between the operating table (or the position where the surgeon may stand) and the second display device 4C are set in advance. In addition, when an installation position of each display device is moved, the distance is updated if the distance after movement is newly input via the input unit 15. In addition, the communication module 53 has a communication means for wirelessly communicating with the second display device 4C.

The optimum pixel number calculation unit 521 calculates the optimum number of pixels of each of the first display device 4B and the second display device 4C based on the limiting resolution and the set distance of each display device.

The output pixel number calculation unit 522 calculates the number of output pixels of each of the first display device 4B and the second display device 4C using each optimum number of pixels.

The display image generation unit 523 generates an image signal for display to be displayed on each display device based on the number of output pixels of each of the first display device 4B and the second display device 4C.

In this manner, the display image is generated based on the number of output pixels individually set for each display device in the second modification so that an image signal with the amount of data corresponding to the distance between the display device and the surgeon is generated.

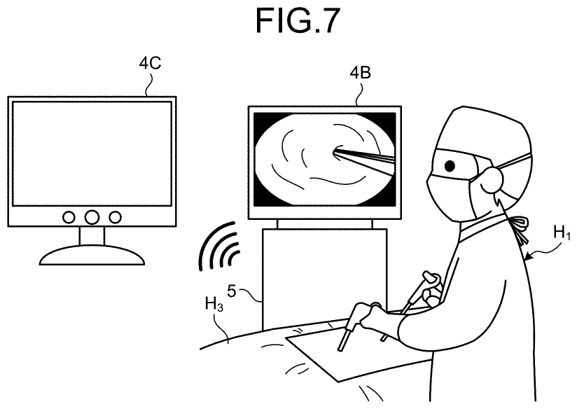

FIG. 7 is a view for describing a use mode of a microscope device of a medical observation system according to the second modification of the first embodiment. The surgeon H₁ performs an operation while observing an image of a surgical site displayed on the first display device 4B or the second display device 4C. In the example illustrated in FIG. 7, the viewing distance corresponds to the distance between the surgeon H₁ and the first display device 4B (first distance) and the distance between the surgeon H₁ and the second display device 4C (second distance).

Here, the first display device 4B is electrically connected to the control device 5 in a wired manner, and the second display device 4C transmits and receives a signal in a wireless manner. For example, the respective display devices are used such that the first display device 4B is the main display device and a second display device 2C is the secondary display device. Since the second display device 2C does not need a cable, the installation position thereof may be freely set. When the installation position of the second display device 2C is far from an observer, an image with a small amount of data to be transmitted may be displayed.

According to the second modification described above, the same effect as that of the first embodiment may be obtained.

Further, according to the second modification, the distance information acquisition unit 42 is made to periodically generate distance information, and output pixels of an image are set each time the distance information is input, so that it is possible to display the image having the resolution that follows the movement of the surgeon.

Incidentally, the second modification may be combined with the first modification, such that a distance information acquisition unit is provided in each display device, and the optimum pixel number calculation unit 521 calculates the optimum number of pixels from distance information acquired by the distance information acquisition unit. As the second modification is combined with the distance measurement of the first modification, it is possible to automatically perform image processing according to the movement of each display device. As a result, for example, even when the display device is moved during an operation, an image may be displayed with an appropriate resolution without inputting and updating the distance.

In addition, the example in which the surgeon H₁ performs the operation while observing the videos of the surgical site displayed on the first display device 4B and the second display device 4C has been described in the second modification, but the surgeon H₁ may observe the video displayed on the first display device 4B, and the assistant H₂ may observe the video displayed on the second display device 4C. In addition, the number of the second display devices 4C is not limited to one, and may be plural. In this case, the surgeon H₁ observes the video displayed on the first display device 4B, and the assistant H₂, a first nurse H₄ (not illustrated) who hands over tools to the surgeon, and a second nurse H₅ (not illustrated) who operates a keyboard or the like connected to the endoscope system 1 may each observe different second display device 4C.

### Second Embodiment

Next, a second embodiment will be described. Although the case of being applied to the endoscope system using a rigid endoscope has been described in the above-described first embodiment, a case of being applied to a flexible endoscope system using a flexible endoscope will be described in the second embodiment. Incidentally, the same configurations as the endoscope system 1 according to the first embodiment will be denoted by the same reference signs, and the detailed description thereof will be omitted.

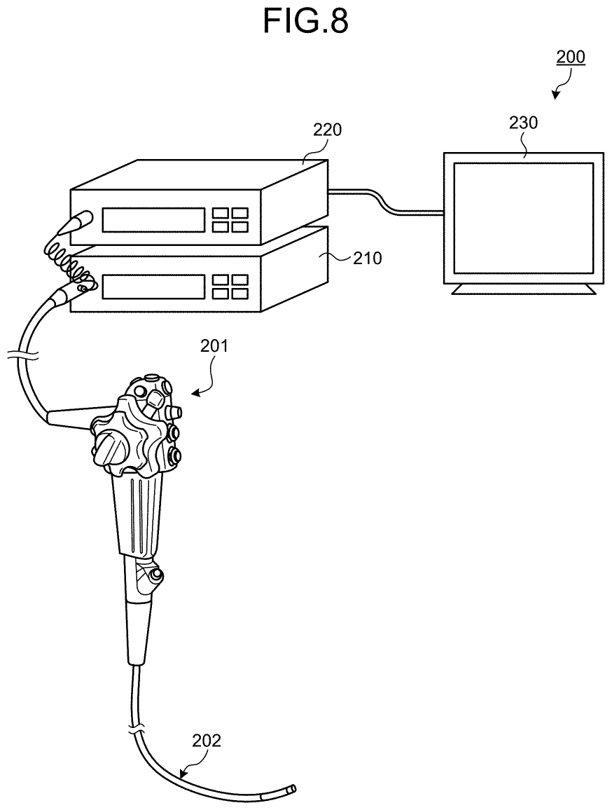

FIG. 8 is a diagram illustrating a schematic configuration of the endoscope system according to the second embodiment. An endoscope system 200 illustrated in FIG. 8 includes: an endoscope 201 that captures an in-vivo image of an observed region by inserting an insertion unit 202 inside a subject and generates image data; a light source device 210 that supplies white light or infrared light to the endoscope 201; a control device 220 that performs predetermined image processing on the imaging signal acquired by the endoscope 201 and collectively controls the operation of the entire endoscope system 200; and a display device 230 that displays the in-vivo image subjected to the image processing by the control device 220.

The endoscope 201 has at least the above-described lens unit 91 and imaging unit 92, and a control unit (corresponding to the camera head controller 94) that controls these.

The light source device 210 includes at least the above-described light source unit 61 and light source controller 62.

The control device 220 includes at least the above-described signal processor 51, image processor 52, communication module 53, input unit 54, output unit 55, control unit 56, and memory 57.

According to the second embodiment described above, the same effect as that of the first embodiment described above may be obtained even with the flexible endoscope system 200.

### Third Embodiment

Next, a third embodiment will be described. Although the endoscope system has been described in the above-described first and second embodiments, a case of being applied to an operating microscope system will be described in the third embodiment. Incidentally, the same configurations as the endoscope system 1 according to the first embodiment will be denoted by the same reference signs, and the detailed description thereof will be omitted.

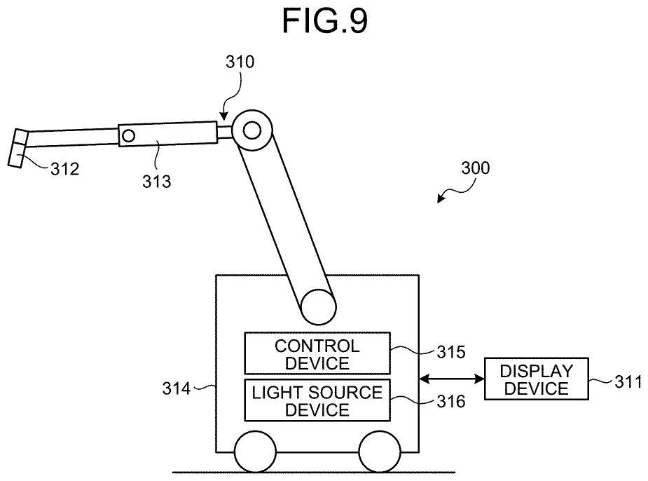

FIG. 9 is a diagram illustrating a schematic configuration of the operating microscope system according to the third embodiment. An operating microscope system 300 illustrated in FIG. 9 is provided with a microscope device 310 which is a medical imaging device that captures and acquires an image to observe a subject, and a display device 311 which displays the image captured by the microscope device 310. Incidentally, the display device 311 and the microscope device 310 may be also integrated.

The microscope device 310 includes: a microscope unit 312 which enlarges and captures a microscopic part of the subject; a support unit 313 which is connected with a proximal end portion of the microscope unit 312 and includes an arm that supports the microscope unit 312 to be rotatable; and a base unit 314 which rotatably holds a proximal end portion of the support unit 313 and is movable on a floor. The base unit 314 includes: a control device 315 that controls an operation of the operating microscope system 300; and a light source device 316 that generates white light, infrared light, or the like to irradiate the subject from the microscope device 310. Incidentally, the control device 315 includes at least the above-described signal processor 51, image processor 52, communication module 53, input unit 54, output unit 55, control unit 56, and memory 57. In addition, the light source device 316 includes at least the above-described light source unit 61 and light source controller 62. In addition, the base unit 314 may be configured to be fixed to a ceiling, a wall surface or the like and support the support unit 313 instead of being provided to be movable on the floor.

The microscope unit 312 has, for example, a columnar shape, and includes the above-described lens unit 91 and imaging unit 92, and a control unit (corresponding to the camera head controller 94) that controls these inside. A switch, which receives input of an operation instruction of the microscope device 310, is provided in a side surface of the microscope unit 312. A cover glass (not illustrated) is provided in an aperture surface of a lower end portion of the microscope unit 312 to protect the inside thereof.

The operating microscope system 300 configured in this manner allows a user such as a surgeon to move the microscope unit 312, perform a zooming operation, and switch the illumination light while operating various switches with the microscope unit 312 being gripped. Incidentally, it is preferable that a shape of the microscope unit 312 be a shape which extends to be elongated in an observation direction to allow the user to easily grip the unit and change a viewing direction. Thus, the shape of the microscope unit 312 may be a shape other than the columnar shape, and may have, for example, a polygonal column shape.

According to the third embodiment described above, the same effect as that of the first embodiment may be obtained even with the operating microscope system 300.

### OTHER EMBODIMENTS

Variations may be formed by appropriately combining a plurality of components disclosed in the medical observation systems according to the first to third embodiments of the present disclosure described above. For example, some components may be deleted from all the components described in the medical observation systems according to the first to third embodiments of the present disclosure described above. Further, the components described in the medical observation systems according to the first to third embodiments of the present disclosure described above may be appropriately combined.

In addition, in the medical observation systems according to the first to third embodiments of the present disclosure, the above-described “unit” may be read as “means” or “circuit”. For example, the control unit may be read as a control means or a control circuit.

In addition, a program to be executed by the medical observation systems according to the first to third embodiments of the present disclosure is provided in the state of being recorded on a computer-readable recording medium, such as a CD-ROM, a flexible disk (FD), a CD-R, a digital versatile disk (DVD), a USB medium, and a flash memory, as file data in an installable or executable format.

In addition, the program to be executed by the medical observation systems according to the first to third embodiments of the present disclosure may be stored on a computer connected to a network such as the Internet and provided by being downloaded via the network.

Although some of the embodiments of the present application have been described in detail with reference to the drawings as described above, these are merely examples, and it is possible to carry out the present disclosure in other forms in which various modifications and improvements have been made based on the knowledge of those skilled in the art, including the aspects described in this disclosure.

Incidentally, the present technique may also have the following configurations.

As described above, the medical image processing device, the medical observation system, and the method of operating the medical image processing device according to the present disclosure are advantageous in terms of suppressing the load related to data processing while maintaining the visibility.

According to the present disclosure, it is possible to achieve an effect that a load related to data processing may be suppressed while maintaining the visibility.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A medical image processing device comprising
an image processor configured to generate a display image based on a distance between a display device and a target and a size of a display screen in the display device, the display image being displayed on the display device, wherein
an image quality of the display image is lowered as the distance becomes larger and the size of the display screen becomes smaller.

2. The medical image processing device according to claim 1, wherein the image processor is configured to:
calculate optimum number of pixels based on the distance and limiting resolution of a human eye;
calculate number of output pixels based on the optimum number of pixels and the size of the display screen; and
generate the display image based on the number of output pixels.

3. The medical image processing device according to claim 1, further comprising
an input device configured to receives input of various types of information,
wherein the distance is input via the input device.

4. The medical image processing device according to claim 2, wherein the image processor is further configured to calculate the distance between the display device and the target based on information on a distance obtained by measuring the distance between the display device and the target.

5. The medical image processing device according to claim 1, wherein the image processor is configured to generate the display image based on a distance to a target located closest to the display device and the size of the display screen.

6. The medical image processing device according to claim 1, wherein the image processor is configured to generate, for each of a plurality of display devices, a display image to be displayed on each of the display devices, based on each distance between each of the display devices and the target and a size of a display screen of each of the display devices.

7. The medical image processing device according to claim 6, wherein among the plurality of display devices, a display device installed at a position far from the target transmits and receives a signal by wireless communication with the medical image processing device.

8. A medical observation system comprising:
the medical image processing device according to claim 1; and
a display device configured to display the display image generated by the medical image processing device.

9. A method of operating a medical image processing device configured to generate a display image displayed on a display device, the method comprising
generating a display image based on a distance between a display device and a target and a size of a display screen in the display device, the display image being displayed on the display device, wherein
an image quality of the display image is lowered as the distance becomes larger and the size of the display screen becomes smaller.
